(19) United States
(12) Patent Application Publication
(10) Pub. No.: US 2012/0064608 A1
(43) Pub. Date: Mar. 15, 2012

(54) METHOD OF CULTURING PHOTOSYNTHETIC ORGANISMS

(75) Inventors: Marc Rene Stammbach, Killara (AU); Peter De Nys, Queensland (AU); Kristen Heimann, Queensland (AU); Alex Rogers, Victoria (AU)

(73) Assignee: MBD Energy Limited, East Melbourne (AU)

(21) Appl. No.: 13/321,284

(22) PCT Filed: Sep. 22, 2009
(86) PCT No.: PCT/AU09/01255

§ 371 Date: Nov. 18, 2011

(30) Foreign Application Priority Data
May 18, 2009 (AU) 2009902229

(51) Int. Cl. C12N 1/12; A01G 1/00; C12M 3/00

(52) U.S. Cl. 435/257.1; 435/292.1; 47/1.4

(57) ABSTRACT

The present invention provides cultivation chambers and methods for the cultivation of photosynthetic organisms, particularly algae.

### FIELD OF THE INVENTION

The present invention relates to cultivation chambers and methods for the production of photosynthetic organisms, in particular microalgae.

### BACKGROUND OF THE INVENTION

The culturing of photosynthetic organisms, particularly microalgae and cyanobacteria, has become the focus of much interest due to the multiple applications for such microorganisms. Firstly, the culturing of photosynthetic microorganisms can utilise waste carbon dioxide (CO₂) and nutrients (for example from sewerage or agriculture outputs) and, in the presence of light, convert these into biomass. Secondly, the produced biomass has the potential for a multitude of uses including: the extraction of oils, which may then be converted into biodiesel; as raw materials for the bioplastics industry; to extract nutraceutical, pharmaceutical and cosmetic products; for animal feed and as feedstock for biodiesel, pyrolysis and gasification plants.

Algae, such as microalgae, may be cultivated in both open and closed systems. The open systems include ponds and raceways or canals and the closed systems include photobioreactors made up of enclosed tubes or other housings which allow light to penetrate to the medium containing the algae.

The open systems have the advantage of generally being cheaper to set up than the closed systems. However, the fact that these systems are open to the environment produces problems with lack of temperature control and potentially a greater risk that the culture becomes contaminated with undesirable organisms. Furthermore, the mixing of the culture medium to maintain the distribution of nutrients and gases may be more difficult in an open pond or raceway system.

While there is greater control over temperature and nutrient supply in closed photobioreactor systems, these types of systems often have the disadvantage of high costs to set up and a lack of flexibility. There are also difficulties in efficiently maintaining a suitable mixing and nutrient/temperature distribution in closed bioreactor systems.

The present invention aims to address one or more of the difficulties of the systems known in the art for culturing photosynthetic organisms, particularly microalgae.

### SUMMARY OF THE INVENTION

In a first aspect, the present invention provides a cultivation chamber for the culture of photosynthetic organisms including:

(a) a wall or walls defining:
a gas space; and
a culture medium containment area below the gas space;

(b) one or more gas inlets positioned within the culture medium containment area such that, in use, gas passes through the culture medium; and

(c) one or more gas outlets in communication with the gas space;

the cultivation chamber permitting exposure of the culture medium to natural light and/or including an artificial light source.

In a further aspect, the present invention provides a method of cultivating photosynthetic organisms including the steps of:

(a) providing a cultivation chamber including:
(i) a wall or walls defining:
   a gas space; and
   a culture medium containment area below the gas space;
(ii) one or more gas inlets positioned within the culture medium containment area such that, in use, gas passes through the culture medium; and
(iii) one or more gas outlets in communication with the gas space;

(b) introducing into the cultivation chamber a culture medium and an inoculate of photosynthetic organisms, the cultivation chamber permitting exposure of the culture medium to natural light and/or including an artificial light source;

(c) introducing gas through the gas inlets and allowing gas to pass out through the gas outlets, wherein the flow of gas thereby mixes the culture medium; and

(d) allowing the photosynthetic organisms to grow in the presence of light.

### DETAILED DESCRIPTION

Gas may be passed into the cultivation chamber via the gas inlets. Where the gas inlets are situated below the surface of the culture medium containing the photosynthetic organisms, gas may be bubbled into the culture medium. The introduction of gas below the surface of the culture medium allows for the mixing of the culture medium and assists in the distribution of the gases, nutrients, light and heat throughout the culture medium. In a preferred embodiment, the gas is introduced in a substantially continuous manner while the photosynthetic organisms are photosynthesising (in the presence of light).

In a preferred embodiment, the gas inlets are positioned along a base portion of the culture medium containment area.

In one embodiment, one or more walls of the cultivation chamber are composed of a flexible material, which may allow for the inflation of the cultivation chamber. Such a flexible material includes, but is not limited to, a plastic-type film. In a preferred embodiment, the cultivation chamber is in the form of an enclosed flexible plastic structure, such as a plastic bag-type structure.

In a preferred embodiment, one or more walls of the cultivation chamber are light-transmissible.

Where the cultivation chamber is inflatable, the inflation of the cultivation chamber may be maintained by the flow of gas achieved through the introduction of gas into the chamber through the gas inlets and out through the gas outlets. The gas may be introduced into the cultivation chamber through one or more gas inlets positioned above and/or below the surface of the culture medium.

Accordingly, in a further aspect, the present invention provides a method of cultivating photosynthetic organisms including the steps of:

(a) providing an inflatable cultivation chamber including:
(i) a wall or walls defining:
   a gas space; and
   a culture medium containment area below the gas space;
(ii) one or more gas inlets positioned within the culture medium containment area such that, in use, gas passes through the culture medium; and
(iii) one or more gas outlets in communication with the gas space;

(b) introducing into the cultivation chamber a culture medium and an inoculate of photosynthetic organisms, the cultivation chamber permitting exposure of the culture medium to natural light and/or including an artificial light source;

(c) introducing gas through the gas inlets and allowing gas to pass out through the outlets, wherein the flow of gas mixes the culture medium and maintains the cultivation chamber in an inflated state; and

(d) allowing the photosynthetic organisms to grow in the presence of light.

Preferably a number of gas inlets are positioned throughout the cultivation chamber. In a preferred embodiment, the gas inlets are positioned at the base of the cultivation chamber, preferably along the length of the base of the cultivation chamber.

In a further preferred embodiment, the gas inlets are positioned along conduits at the base of the cultivation chamber, wherein the conduits are adapted to carry and distribute the flow of gas. The gas inlets are preferably positioned along the conduits at intervals that allow for a substantially even distribution of gas flow along the length of an elongated cultivation chamber.

In a further embodiment, the gas outlets are designed to release excess gas pressure that may build up in the flexible cultivation chamber.

In a preferred embodiment, the gas outlets may include a valve system, preferably a one-way valve system. The use of one-way valves may reduce the risk of contamination of the cultivation chamber from outside air.

In a further embodiment, gas is passed into the cultivation chamber through gas inlets both above the surface of the culture medium and below the surface of the culture medium. The introduction of the gas above the surface of the culture medium allows for a modification of the atmosphere in the cultivation chamber.

Suitable gases and/or liquid nutrients may be introduced into the cultivation chamber of the present invention to aid the growth of the photosynthetic organisms. Such gases or liquids may be selected from carbon dioxide (CO₂); fertilisers and waste from aquaculture and agriculture (for example: trout, salmon, cattle, pig and chicken farms). The CO₂ may be from any suitable source and may be from air or in a concentrated form. Examples of suitable concentrated sources of CO₂ include, but are not limited to, flue gases, kiln and incineration gases and gases from anaerobic digestion. In a preferred embodiment, the source of CO₂ is a flue gas, more preferably desulphurised flue gas (DFG).

The concentration of CO₂ introduced into the cultivation chamber may be varied by varying the amount of air mixed with the CO₂. For example, CO₂-containing flue gas may be diluted with air, depending on the CO₂ requirements of the photosynthetic organisms. During periods of darkness, for example at night when natural light is used, the amount of CO₂ may be decreased while maintaining a constant gas flow by increasing the amount of air in the mixture. The air to be mixed with the CO₂ source may be filtered to remove certain particulate matter, for example using a particulate air filter, more preferably a high efficiency particulate air (HEPA) filter.

The culture medium may be any suitable medium for the growth of the desired photosynthetic organisms. The culture medium may be based on fresh or saline water and may include waste water from industrial processes or sewerage treatment systems.

A source of light is required for the organisms to photosynthesise. Any suitable source of light may be used including natural light and artificial light or a combination of natural and artificial light. Artificial light may be provided by any suitable light source. In one embodiment, the artificial light source is provided by light-emitting diodes (LEDs). An artificial light source may be provided to extend the length of time per day that the organisms continue to photosynthesise beyond daylight hours. Accordingly, in one embodiment, the cultivation chambers and methods of the present invention are adapted to alternate between using natural and artificial light as required.

In a further aspect, the present invention provides a method of cultivating photosynthetic organisms including the steps of:

(a) providing a cultivation chamber including:
(i) a wall or walls defining:
   a gas space; and
   a culture medium containment area below the gas space;
(ii) one or more gas inlets and one or more gas outlets in communication with the gas space; and
(iii) a gas flow control means;

(b) introducing into the cultivation chamber a culture medium and an inoculate of photosynthetic organisms, the cultivation chamber permitting exposure of the culture medium to natural light and/or including an artificial light source;

(c) controlling the flow of gas in through the gas inlets and out through the gas outlets using the gas flow control means, wherein the flow of gas drives evaporation from the culture medium; and

(d) allowing the photosynthetic organisms to grow in the presence of light.

The gas flow control means is preferably a fan. The fan is preferably situated at one end of an elongate cultivation chamber.

In a preferred embodiment, the cultivation chamber is inflatable. In a further embodiment the flow of gas maintains the cultivation chamber in an inflated state.

It has been found that evaporation from the culture medium may enable a control of the temperature within the cultivation chamber. This assists in the culture of the microorganisms, as temperature control is important to gaining optimal growth and/or optimal production of the relevant chemicals by the microorganisms (such as triglycerides as ingredients for biodiesel).

To counteract any unwanted salinity increases associated with the evaporation of the water from the culture medium, additional water may be added to the culture medium. This additional water may be in any suitable form, for example as fresh water or aquaculture waste water.

A further mechanism for controlling the temperature of the culture medium using the method of the present invention is to control the rate at which gas is introduced into the cultivation chamber and/or the temperature of the introduced gas. For example, heat loss at lower than optimal ambient temperatures may be reduced by lowering the amount of gas at a temperature lower then ambient temperature being introduced into the culture medium, thereby reducing the mixing of the medium and resultant heat exchange. Accordingly, in darkness the gas flow may be reduced and may be stopped completely to at least partially maintain the temperature of the cultivation medium during low night time ambient temperatures.

Furthermore, by varying the temperature and composition of the gas introduced into the cultivation chamber the temperature of the liquid culture medium may be varied. For example, if using enriched CO₂ from flue gas as an input, the flue gas may be maintained at a higher temperature to counteract the effect of low ambient temperatures. Accordingly, the flue gas may be introduced at a higher temperature where the temperature of the culture medium needs to be increased. Conversely, where the temperature of the liquid culture medium needs to be reduced, the flue gas may be cooled further before introducing to the cultivation chamber.

Alternatively, increasing the amount of air introduced into the cultivation chamber may aid in the cooling of the cultivation medium. This air may be introduced by bubbling from under the surface of the cultivation medium or by being passed over the surface of the cultivation medium.

Alternatively, the temperature of the cultivation medium may be controlled by circulating it directly or indirectly over a suitable heat exchanger, such a cooling tower or a boiler.

The cultivation chamber may be of any suitable size to cultivate the required amount of the photosynthetic organisms.

In a preferred embodiment, the cultivation chamber may be about 1 metre to about 10 metres, more preferably about 2 metres to about 6 metres in width. In a particularly preferred embodiment, the cultivation chamber of the present invention is about 3 metres in width.

In a further preferred embodiment, the cultivation chamber is about 5 metres to about 250 metres, more preferably about 10 metres to about 100 metres in length. In a particularly preferred embodiment, the cultivation chamber is about 50 metres in length.

The culture medium can be present in the cultivation chamber in any suitable volume for the optimal cultivation of the photosynthetic organisms. In a preferred embodiment, the volume of culture medium is such that it is present in the cultivation chamber to a depth of about 20 centimetres to about 120 centimetres, more preferably about 30 centimetres to about 100 centimetres. In a particularly preferred embodiment, the culture medium is at a depth of about 60 centimetres in the cultivation chamber.

In one embodiment, the level of the culture medium in the cultivation chamber is controlled by the regulation of the intake of culture medium through one or more liquid ports, which act as inlets and/or outlets for the passage of liquid in to and out of the cultivation chamber. The passage of the culture medium through the liquid ports in one or both directions is preferably regulated by one or more valves, which are reactive to the level of the culture medium in the cultivation chamber, thereby allowing for the emptying (for example, for harvesting the photosynthetic organisms) and refilling of the cultivation chamber. In a preferred embodiment the valves are ball valves.

In a further embodiment, the level of the culture medium in the cultivation chamber is measured by means of one or more pressure sensors.

The photosynthetic organisms may be selected from any suitable organisms and may be cultured as a single species in monoculture or two or more species in the same cultivation chamber. Photosynthetic organisms that produce useful ingredients for the chemical, biodiesel, pharmaceutical or nutraceutical industries are preferred. Suitable photosynthetic microorganisms include cyanobacteria (blue-green algae) and algae, preferably microalgae. The microorganisms may grow in fresh or salt water. Examples of photosynthetic microorganisms that may produce useful ingredients/feedstocks include, but are not limited to, those belonging to the following genera: Chlamydomoas; Chaetoceros; Cladophora; Chaetomorpha; Dunaliella; Haematococcus; Isochrysis; Nannochloropsis; Porphyridum; Picochlorum (synonym Nannochloris); Pleurochrysis;, Rhodomoas, Spriulina. The method of the present invention may also be utilised to culture macroalgae, for example those of the genus Ulva.

The photosynthetic organisms produced according to the present invention have a number of potential uses. Oil (e.g. triglycerides) may be extracted from the microorganism and this oil may be used for: biodiesel production (e.g., using known transesterification processes); as a raw material for the production of plastics and for the synthesis of jet and other fuels. The cake component of the biomass that is left after the extraction of oil may be used as: feed for the livestock industry; fertilizer production; biomass for bio-plastic production or biomass for energy production and/or pyrolysis. Photosynthetic organisms may also produce other useful products, such as nutraceuticals (e.g. omega 3 and 6 fatty acids; antioxidants, such as astaxanthin and pigments, such as β-carotene), phycocolloids, triglycerides and other ingredients for the pharmaceutical and cosmetics industries.

Accordingly, in a further aspect the present invention provides a product extracted from photosynthetic organisms produced in accordance with the method of the present invention. In one embodiment, the product is selected from the group consisting of an oil; glycerol; omega 3 and 6 fatty acids; astaxanthin; and β-carotene. In another embodiment, the product is biomass cake, such as algae cake.

Where the cultivation chamber is inflatable, gas outlets may be provided above the level of the cultivation medium to release the pressure that is built up through the gas which has been bubbled through the cultivation medium, for example from the base of the cultivation chamber.

Accordingly, in a further aspect, the present invention provides an inflatable cultivation chamber for the culturing of photosynthetic organisms including:

(a) a wall or walls defining:
a gas space; and
a culture medium containment area below the gas space;

(b) one or more gas inlets positioned within the culture medium containment area and one more gas outlets in communication with the gas space;

(c) wherein, in use, gas passes through the culture medium and into the gas space causing the chamber to inflate; and

(d) wherein, in use, excess gas passes out through the gas outlets;

the cultivation chamber permitting exposure of the culture medium to natural light and/or including an artificial light source.

In one embodiment, the cultivation chamber includes one or more liquid ports to allow for the introduction and removal of the culture medium. Preferably, the one or more liquid ports include a regulation means to control the introduction and removal of the culture medium from the cultivation chamber. More preferably, the regulation means is a valve, such as a ball valve.

The cultivation chambers of the present invention may be joined together to form a series of cultivation chambers in a continuous system for the cultivation of the photosynthetic organisms. Any suitable number of cultivation chambers may be connected via a manifold, whereby the movement of culture medium and gas may be centrally regulated for each manifold. The central regulation may be achieved, for example, by an automated valve that controls the flow of the culture medium. A central regulation of the level of culture medium in the cultivation chambers may also be achieved by using one or more pressure sensors to monitor the level of the culture medium, which can then allow an automated response to fill or empty the cultivation chambers to the required level.

A manifold may be utilised to flow connect any suitable number of cultivation chambers according to the present invention. Preferably 10 to 200 cultivation chambers may be joined; more preferably 20 to 60.

Photosynthetic organisms convert carbon dioxide, water and nutrients into biomass in the presence of light. Therefore, the growth of these photosynthetic organisms enables carbon dioxide emitted as, for example, flue gas from a power plant, refinery or cement kiln, liquid natural gas production or coal seam gas, to be recycled as biomass rather than being released into the atmosphere.

Accordingly, in a further aspect the present invention provides a method for the conversion of carbon dioxide to algal biomass including the steps of:

(a) providing a cultivation chamber including:
(i) a wall or walls defining:
   a gas space; and
   a culture medium containment area below the gas space;
(ii) one or more gas inlets positioned within the culture medium containment area such that, in use, gas passes through the culture medium; and
(iii) one or more gas outlets in communication with the gas space;

(b) introducing into the cultivation chamber a culture medium and an inoculate of algae, the cultivation chamber permitting exposure of the culture medium to natural light and/or including an artificial light source;

(c) introducing carbon dioxide-containing gas through the gas inlets and allowing excess gas to pass out through the gas outlets, wherein the flow of gas thereby produced mixes the culture medium; and

(d) allowing the algae to grow in the presence of light to produce algal biomass.

In a further aspect, the present invention provides a method of recycling emitted carbon dioxide by utilising the emitted carbon dioxide as an input in the production of photosynthetic organisms using the method of the present invention. The emitted carbon dioxide may be flue gas, kiln gas, incineration gas and gas from anaerobic digestion.

Where the CO₂ is provided from flue gas, the flue gas is preferably cooled and partly scrubbed of pollutants such as SOx and NOx, dust, heavy metals etc before it is introduced into the cultivation chamber. Heavy metals, SOx and NOx and dust remaining in the flue gas after partial scrubbing may provide micronutrients required for the growth of the photosynthetic organism. Such micronutrients may then be added to the culture medium, either directly or with additional treatment, e.g. the selective removal of heavy metals.

In the figures:

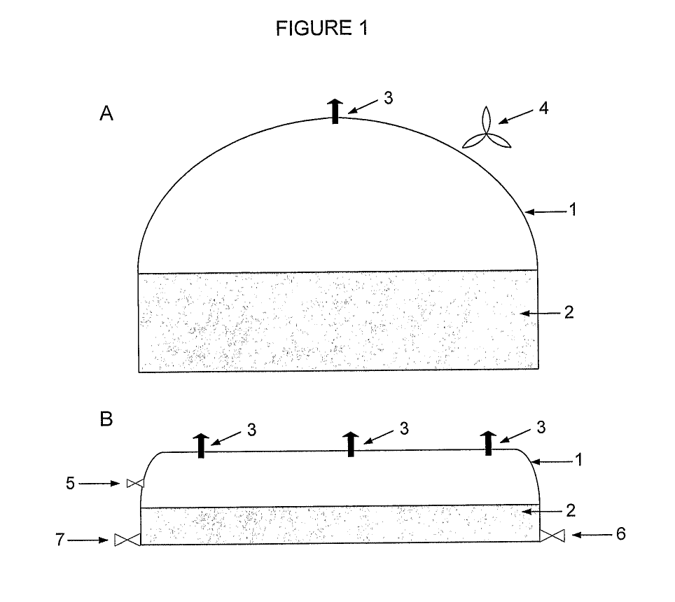

FIG. 1: (A) Bag cultivation chamber 1 front view and (B) side view, including: a flexible bag (1) containing a culture medium for growing algae (2); gas outlet (3); fan (4); gas inlet (5); cultivation medium outlet (6); and cultivation medium inlet (7).

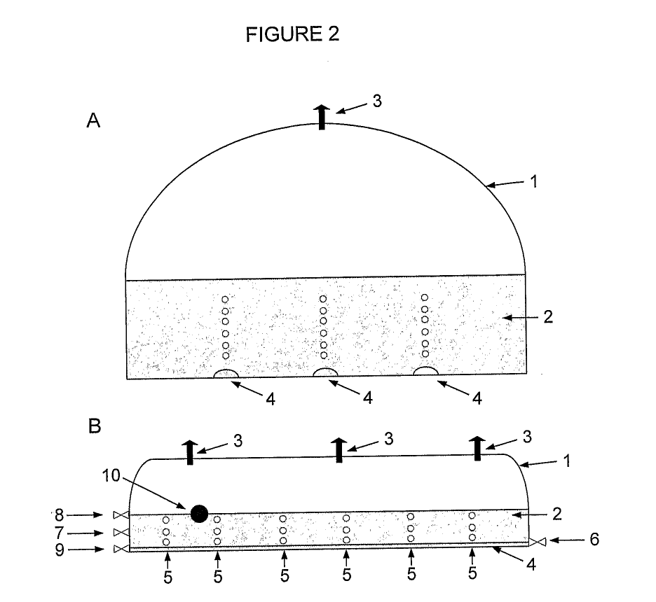

FIG. 2: (A) Bag cultivation chamber 2 front view and (B) side view, including: a flexible bag (1) containing a culture medium for growing algae (2); gas outlet (3);gas bubbling tracks (4) with pinprick holes (5); gas inlets (6); cultivation medium outlet (7); cultivation medium inlet (8); draining outlet (9) and ball valve (10) to regulate ports (7), (8) and (9).

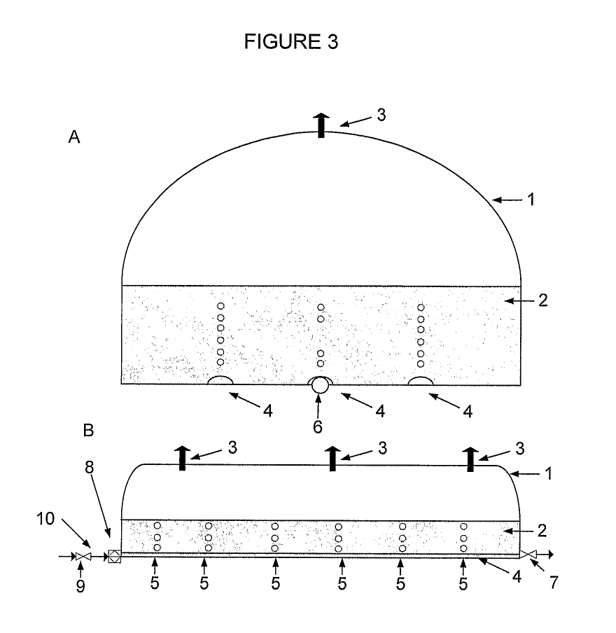

FIG. 3: (A) Bag cultivation chamber 3 front view and (B) side view, including: a flexible bag (1) containing a culture medium for growing algae (2); gas outlet (3);gas bubbling tracks (4) with pinprick holes (5); gas inlets (6); cultivation medium outlet (7); cultivation medium inlet (10) with pressure sensor (8) and ball valve.

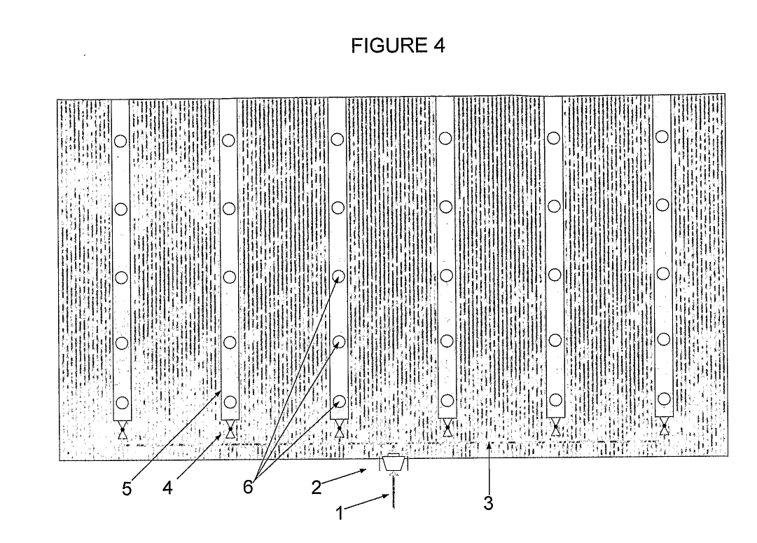

FIG. 4: Top view of gas bubbling tracks (5) in the base of a bag cultivation chamber having a gas inlet (1) with compression fitting (2), a conduit (3) to transport the gas to restrictive flow orifices (4) and the end of each track (5) and pinprick holes (6) to allow the exit of gas.

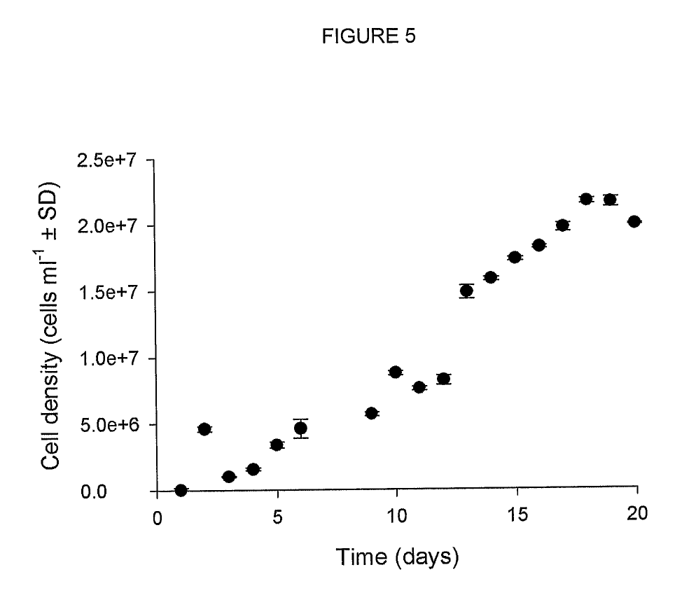

FIG. 5: Cell density (cells mL-1) from day 1 (inoculation) to day 20. Average ±standard deviation, n=3.

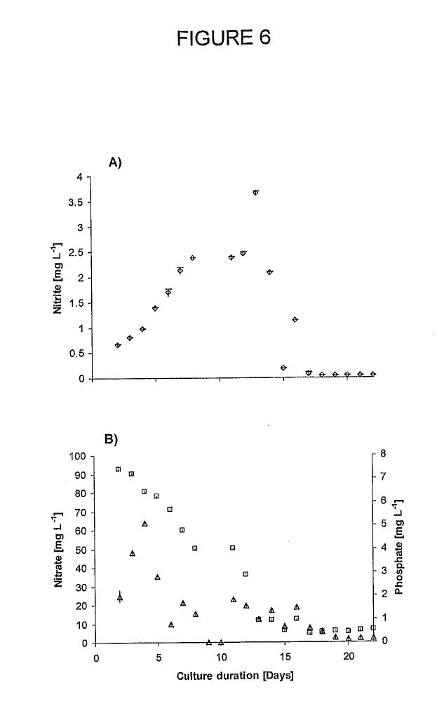

FIG. 6: Time course of nutrient concentrations in the bag cultivation chamber. A) nitrite, B) nitrate (red squares) and phosphate (black triangles). Average ±standard deviation, n=3.

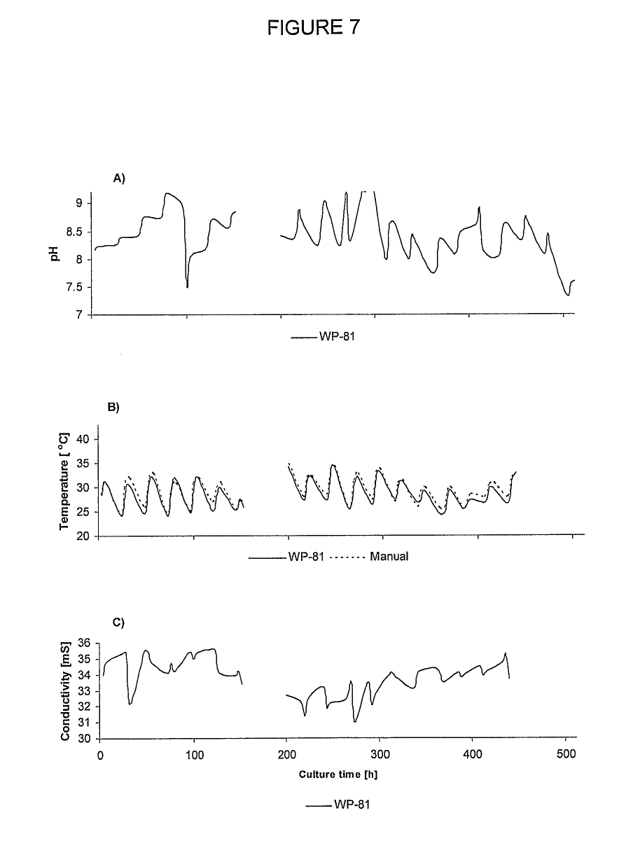

FIG. 7: Fluctuation of A) pH, B) temperature, and C) conductivity over culture time of Nannochloropsis oculata in the bag. WP-81: handheld TPS pH- and conductivity-meter, manual: handheld thermometer.

### EXAMPLE 1

A chamber for the cultivation of photosynthetic organisms was created using a bag culture system as shown in FIG. 1.

The operation of bag cultivation chamber 1 is as follows:

1. A fan (4) inflates the empty cultivation chamber (without culture medium (2)) to operational volume, with all excess pressure exiting through the gas outlet (3). The fan is continuously running so as to ensure the bag (1) does not deflate.

2. The empty cultivation chamber is inoculated with 10 000 l of microalgae culture (0.2% algae) produced in a separate photobioreactor and topped up with 10 000 l filtered and treated recycled saline waste water.

3. CO₂ is injected continuously during daylight hours through the gas inlet (5). The microalgae absorb the required quantities of CO₂ and the excess is released through the always open gas outlet (3).

4. An additional 20 000 l of recycled saline waste water is added, bringing the total capacity to 40 000 l of culture medium.

5. This process continues for another 24 hours until total harvesting capacity reaches 100 000 l. At this stage, the level of the culture medium (2) in the cultivation chamber is 60 cm.

6. After the algae have reached maximum harvest capacity (72 hours), 50 0000 l is harvested from the cultivation medium outlet (6).

7. 50 000 l of recycled saline waste water is returned to the cultivation chamber via the cultivation medium inlet (7), bringing the total culture medium volume back to 100 000 l.

8. The harvesting and return cycle repeats once every 24 hours, while maintaining continuous CO₂ injection during daylight hours.

### EXAMPLE 2

The bag cultivation chamber described in Example 1 was modified as shown in FIG. 2.

The operation of bag cultivation chamber 2 is as follows:

1. Cultivation chamber 2 inoculation follows the same procedure as Example 1 steps 2, 4 and 5 to bring the harvesting capacity to 100 000 l within 72 hours.

2. CO₂ is pre-mixed with a high efficiency particulate air (HEPA) filtered air stream and fed through gas inlets (6) to gas bubbling tracks (4). These tracks are pinpricked (5) at suitable intervals to allow even air/CO₂ distribution along the length of the bag cultivation chamber. This bubbling operates continuously, with the CO₂ component reduced overnight.

The air/CO₂ injection acts to slowly inflate the bag (1) and maintain circulation of the algae in the culture medium (2). Excess pressure is released through the one-way valve regulated gas outlet (3). This creates a closed loop system to minimise the contamination risk.

3. After the 72 hours of culturing the microalgae, 50 000 l is harvested from the ball-valve-regulated (10) cultivation medium outlet (7), which is positioned at 30 cm in height. Once the cultivation medium reaches 30 cm, a signal is sent to the automation system that the cultivation chamber is at 50 000 l capacity.

4. 50 000 l of treated recycled saline waste water is returned to the cultivation chamber via the ball valve-regulated (10) cultivation medium inlet (8), sending a back pressure signal to the automation system that the cultivation chamber is now at 100 000 l.

5. The ball valve-regulated (10) draining outlet (9) allows the complete draining of cultivation chamber in case of contamination or for a regular cleaning routine. The remaining cultivation medium is either drained to the harvesting system for processing or, in the case of contamination, to the UV treatment system.

### EXAMPLE 3

The bag cultivation chamber described in Example 1 was further modified as shown in FIG. 3.

The operation of bag cultivation chamber 3 is as follows:

1. Cultivation chamber 3 inoculation follows the same procedure as Example 1 steps 2, 4 and 5 to bring the harvesting capacity to 100 000 I within 72 hours.

2. CO₂ is pre-mixed with a high efficiency particulate air (HEPA) filtered air stream and fed through gas inlets (6) to gas bubbling tracks (4). These tracks are pinpricked (5) at suitable intervals to allow even air/CO₂ distribution along the length of the cultivation chamber. This bubbling operates continuously, with the CO₂ component reduced overnight.

The air/CO₂ injection acts to slowly inflate the bag (1) and maintain circulation of the algae in the culture medium (2). Excess pressure is released through the one-way valve regulated gas outlet (3). This creates a closed loop system to minimise the contamination risk.

3. After the 72 hours of culturing the microalgae, 50 000 l is harvested from the cultivation chamber through the ball valve-regulated harvesting outlet (7). The required volume is determine by measuring volume in reference to a pressure head sensor (8).

4. 50 000 l of treated recycled saline waste water is returned to the cultivation chamber via the ball valve-regulated (9) cultivation medium inlet (10) with pressure head sensor (8), sending a back pressure signal to the automation system that the cultivation chamber is now at 100 000 l.

Further detail (in top view) of a gas bubbling setup that may be included in the modified cultivation chamber is provided in FIG. 4. This figure shows six gas bubbling tracks (5) with pinprick holes (6) which are fed with gas introduced through the gas inlet (1) and compression fitting (2) via a gas conduit (3) and restrictive flow orifices (4) at the end of each track. The restrictive flow orifices serve to divide the gas flow evenly between the air distributor vanes. The flow rate of the gas through the gas inlet is approximately 100 kg/hr and through the restrictive flow orifice 17 kg/hr.

### EXAMPLE 4

The growth of the microalga Nannochloropsis oculata was tested using the bag cultivation chamber described in Example 1. This culture bag was 10 m in length and 3 m in width and fitted with a six-bladed fan at one end to keep the bag inflated and drive evaporation. Along the top of the bag, four holes (13 cm diameter) allowed hot air and vapor to escape. This evaporation assisted in maintaining the algal culture at more stable temperatures.

In this trial, both freshwater and filtered marine aquaculture waste (A3) water was added to the culture to account for salinity increases and evaporative loss of liquid. The bag cultivation chamber was filled to approximately 0.30 m in depth, resulting in a final culture volume of slightly less than 9 m³. The algae were cultivated in sea water than had been filtered through 20 μm, 5 μm and 1 μm filters.

Aeration and CO₂ enrichment was provided through tubing designed for the gas diffusion via delivery into liquid media. This tubing had an outer diameter of 25 mm, an inner diameter of 10 mm and a porous wall of 7.5 mm thickness.

The bag cultivation chamber system was inoculated with Nannochloropsis oculata to a comparatively low cell density of 2.1×10⁴ cells mL⁻¹ and not filled up to full capacity volume. Already after 24 h, cell densities had increased dramatically, and the bag was filled up to its maximum depth on day 2. The growth of the culture up to the harvest of the algae on day 20 is shown in FIG. 5.

### Nutrient Consumption

There was a steady increase in nitrite from the day of inoculation (0.5 mg L⁻¹) to day 8 (2.5 mg L⁻¹) (FIG. 6 A). After a few days at a steady concentration, nitrite peaked at 3.7 mg L⁻¹ on day 13, and then was rapidly utilized. Within a few days, nitrite was depleted and remained so until the culture crashed. Nitrate was a high 90 mg L⁻¹ at the beginning of the period, and was steadily being utilized (FIG. 6 B). From day 13, nitrate concentration remained stable around 10 mg L⁻¹. There was an increase in phosphate the first few days (through addition of filtered A3 water to top the system up) (FIG. 6 B). From day 3, phosphate was being noticeably assimilated and fluctuated between 2 mg L⁻¹ and totally deplete. No nutrients were added to the bag system, however fresh filtered A3 water was regularly added along with freshwater to compensate for evaporation. The added A3 water accounts for the regular, small increases in nutrient concentrations.

### Physical and Chemical Parameters

In the culture, pH quickly rose to over 9 in the first three days (FIG. 7). After day 3, a CO₂ supply was connected and pH could now be regulated by adding CO₂ when a value above 8.4 was recorded.

Photosynthetic activity was high in the bag in the beginning of the period, with rapid changes in pH due to uptake of CO₂ during photosynthesis, leading to large fluctuations in pH.

Temperature fluctuated in diel rhythm, with the highest temperatures measured in the afternoon (4 pm) (FIG. 7 B). Similar to the tank system, temperatures rarely rose above 30° C., and were quite stable.

Conductivity in the bag fluctuated between 32 and 36 mS due to evaporation, and both freshwater and additional filtered A3 water was regularly added (FIG. 7 C).

It will be understood that the invention disclosed and defined in this specification extends to all alternative combinations of two or more of the individual features mentioned or evident from the text or drawings. All of these different combinations constitute various alternative aspects of the invention.

1.-25. (canceled)

26. A cultivation chamber for the culture of photosynthetic organisms including:
(a) a wall or walls defining:
a gas space; and
a culture medium containment area below the gas space;
(b) one or more gas inlets positioned within the culture medium containment area along a base portion of the culture medium containment area. (2) such that, in use, gas passes through the culture medium; and
(c) one or more gas outlets in communication with the gas space;
the cultivation chamber being inflatable and permitting exposure of the culture medium to natural light and/or including an artificial light source.

27. A cultivation chamber according to claim 26, wherein the photosynthetic organisms are selected from the group consisting of macroalgae, microalgae and cyanobacteria.

28. A cultivation chamber according to claim 27, wherein the photosynthetic organisms are microalgae.

29. A cultivation chamber according to claim 26, the chamber being elongate and further including conduits adapted to carry and distribute the flow of gas along at least a portion of the length the cultivation chamber.

30. A cultivation chamber according to claim 26, the gas outlets including a valve system designed to release excess gas pressure from within the chamber.

31. A cultivation chamber according to claim 26, further including one or more liquid ports which act as inlets and/or outlets for the passage of liquid into and out of the cultivation chamber to allow for the harvesting and refilling of the cultivation chamber.

32. A cultivation chamber system for the cultivation of photosynthetic organisms including two or more flow connected cultivation chambers according to claim 31.

33. A method of cultivating photosynthetic organisms including the steps of:
(a) providing an inflatable cultivation chamber including:
(i) a wall or walls defining:
a gas space; and
a culture medium containment area below the gas space;
(ii) one or more gas inlets positioned within the culture medium containment area positioned along the base of the culture medium containment area.
such that, in use, gas passes through the culture medium; and
(iii) one or more gas outlets in communication with the gas space;
(b) introducing into the cultivation chamber a culture medium and an inoculate of photosynthetic organisms, the cultivation chamber permitting exposure of the culture medium to natural light and/or including an artificial light source;
(c) introducing gas through the gas inlets and allowing gas to pass out through the gas outlets, wherein the flow of gas thereby mixes the culture medium; and
(d) allowing the photosynthetic organisms to grow in the presence of light.

34. A method according to claim 33, wherein the flow of gas maintains the cultivation chamber in an inflated state.

35. A method according to claim 34, wherein the gas outlets are designed to release excess gas pressure from within the chamber.

36. A method according to claim 33, wherein the introduced gas includes carbon dioxide.

37. A method according to claim 36, wherein the carbon dioxide-containing gas is from a source selected from the group consisting of flue gas, kiln gas, incineration gas and gas from anaerobic digestion.

38. A method according to claim 33, wherein the gas is introduced in a substantially continuous manner while the organisms are photosynthesising.

39. A method according to claim 33, wherein the photosynthetic organisms are selected from the group consisting of macroalgae, microalgae and cyanobacteria.

40. A method according to claim 33, wherein the culture medium is saline and/or waste water.

41. A method for the conversion of carbon dioxide to algal biomass including the steps of:
(a) providing an inflatable cultivation chamber including:
(i) a wall or walls defining:
a gas space; and
a culture medium containment area below the gas space;
(ii) one or more gas inlets positioned within the culture medium containment area positioned along the base of the culture medium containment area. such that, in use, gas passes through the culture medium; and
(iii) one or more gas outlets in communication with the gas space;
(b) introducing into the cultivation chamber a culture medium and an inoculate of algae, the cultivation chamber permitting exposure of the culture medium to natural light and/or including an artificial light source;
(c) introducing carbon dioxide-containing gas through the gas inlets and allowing excess gas to pass out through the gas outlets, wherein the flow of gas thereby produced mixes the culture medium; and
(d) allowing the algae to grow in the presence of light to produce algal biomass.

42. A method according to claim 41 wherein the source of carbon dioxide is from a source selected from the group consisting of flue gas, kiln gas, incineration gas and gas from anaerobic digestion.

43. A method according to claim 42, wherein the algae are microalgae.

44. A method of cultivating photosynthetic organisms including the steps of:
(a) providing an inflatable cultivation chamber including:
(i) a wall or walls defining:
a gas space; and
a culture medium containment area below the gas space;
(ii) one or more gas inlets positioned within the culture medium containment area positioned along the base of the culture medium containment area. and
(iii) a gas flow control device;
(b) introducing into the cultivation chamber a culture medium and an inoculate of photosynthetic organisms, the cultivation chamber permitting exposure of the culture medium to natural light and/or including an artificial light source;
(c) controlling the flow of gas in through the gas inlets and out through the gas outlets using the gas flow control device, wherein the flow of gas drives evaporation from the culture medium; and
(d) allowing the photosynthetic organisms to grow in the presence of light.
